(19) United States
(12) Patent Application Publication
(10) Pub. No.: US 2023/0414356 A1
(43) Pub. Date: Dec. 28, 2023

(54) DEVICE FOR HEART REPAIR

(71) Applicant: CARDIOMECH AS, Trondheim (NO)

(72) Inventors: Nikolai HIORTH, Oslo (NO); Hans Emil HIORTH, Trondheim (NO)

(21) Appl. No.: 18/036,783

(22) PCT Filed: Nov. 12, 2021
(86) PCT No.: PCT/EP2021/081513

§ 371 Date: May 12, 2023

(30) Foreign Application Priority Data
Nov. 13, 2020 (GB) 2017930.5

(51) Int. Cl. A61F 2/24; A61B 17/04

(52) U.S. Cl. CPC A61F 2/2466; A61F 2/2457; A61B 17/0467; A61M 25/0147

(57) ABSTRACT

A catheter for insertion into the body, a method of use of a catheter for insertion into the body, and a method of manufacture of a catheter for insertion into the body are described. A catheter (1) for insertion into the body comprises: a flexible body portion (2) having a longitudinal axis extending from a proximal end of the catheter (1) towards a distal end of the catheter (1); and a tensile member (3) comprising a first portion (3a) extending along a first length of the tensile member (3) and a second portion (3b) extending along a second length of the tensile member (3), the tensile member (3) in communication with and extending along an outer part of the flexible body portion (2); wherein the tensile member (3) is configured to follow a curvature of the flexible body portion (2) when the catheter (1) is curved; wherein the first portion (3a) is configured to increase a longitudinal stiffness of the catheter (1); and wherein the second portion (3b) is slack along its length when the flexible body portion (2) is in the unconstrained position, such that a longitudinal stiffness of the second portion (3b) is reduced relative to the first portion (3a) until the slack has been taken up through flexing of the catheter (1).

The present invention relates to a catheter for insertion into the body, a method of use of a catheter for insertion into the body, and a method of manufacture of a catheter for insertion into the body.

Catheters are generally thin tubes made of medical-grade materials, used for introducing components or fluids into the body during various medical procedures. To follow various paths, vessels or the like within the body, catheters are usually flexible. For example, WO 2020/109599 discloses a catheter comprising composite tubing embedded in a soft polymer. A wire is also disposed along the length of the shaft to increase the tensile strength of the shaft. The wire generally increases a longitudinal stiffness of the catheter, such that it will return to a rest length and/or elastically resist extension when a tensile force is applied to the catheter. To keep a central passage, or hollow, of the catheter clear the wire is usually located in the embedded polymer. Such catheters may be used in catheter devices for heart repair, such as those discussed in WO 2016/042022 and WO 2020/109599.

Whilst benefits are provided by the wire increasing the tensile strength of the catheter, the provision of the wire may produce an undesirable whipping effect in the catheter, particularly when the catheter is curved.

When the catheter is curved, and hence the composite tubing is curved, a varying radius of curvature for the composite tubing may be observed. The radius of curvature may be measured from a centre point of the curvature. For example, the longitudinal axis of the catheter may define a global radius of curvature for the catheter. However, given that the catheter has a cross-sectional area, the radius of curvature will vary across this cross-section, and hence across the outer surface of the catheter. An inner radius of curvature may be defined by the part of the outer surface which is closest to the centre point of the curvature. An outer radius of curvature may be defined by the part of the outer surface which is furthest from the centre point of the curvature. The outer radius of curvature will comprise a greater path length than an inner radius of curvature, as a result of the difference between the respective radii of curvature.

When the catheter is curved the wire, which is embedded in the wall of the catheter, will follow a path length according to its location relative to the radius of curvature of the respective part of the catheter wall it is embedded in. Due to in particular the longitudinal stiffness of the wire, a greater force may be exerted on the catheter by the wire when the tensile wire follows any path length greater than the path length of the inner radius of curvature. That is, the inner radius of curvature defines the shortest path length the wire may follow for a given curvature of the catheter. Accordingly, this is the most energetically stable, and hence desirable, position for the wire to be located at when the catheter is curved.

The wire may therefore exert a whipping force, a whipping stress and/or a whipping inertia on the catheter. This may result in the catheter rotating such that the wire is located at the inner radius of curvature. This rotation is generally sudden and is undesirable, as it may alter a position of the component introduced into the body by the catheter, or may damage surrounding tissues when the catheter is inserted into the body. The whipping effect may also destabilise a position of the catheter due to the whipping effect produced.

It is therefore an objective of the discussed embodiments to provide an improved catheter.

Viewed from a first aspect of the present invention, there is provided a catheter for insertion into the body, the catheter comprising: a flexible body portion having a longitudinal axis extending from a proximal end of the catheter towards a distal end of the catheter; and a tensile member comprising a first portion extending along a first length of the tensile member and a second portion extending along a second length of the tensile member, the tensile member in communication with and extending along an outer part of the flexible body portion; wherein the tensile member is configured to follow a curvature of the flexible body portion when the catheter is curved; wherein the first portion is configured to increase a longitudinal stiffness of the catheter; and wherein the second portion is slack along its length when the flexible body portion is in the unconstrained position, such that a longitudinal stiffness of the second portion is reduced relative to the first portion until the slack has been taken up through flexing of the catheter.

The tensile member may increase an overall strength of the catheter. In particular, the tensile member may increase a tensile strength of the catheter by increasing a longitudinal stiffness of the catheter. The tensile member will be understood to have a greater longitudinal stiffness than the flexible body portion. The tensile member may decrease extension or elongation of the catheter when the catheter experiences a tensile force, or a load acting from opposing ends of the catheter. The provision of the tensile member along the length of the flexible body portion may distribute forces which actuate the catheter more evenly over its length. This may prevent undesirable effects as a result of uneven stresses across the catheter, such as localised deformations of the flexible body portions which may cause pinching and/or radial compression of the flexible body portion. The catheter may be more durable as a result of the use of the tensile member.

When the catheter is moved, including curved, the flexible body portion alone may extend or elongate and may not be longitudinally stiff enough such that it resists elongation and/or returns to an unconstrained position (i.e. an unconstrained, initial length), either in a short amount of time or at all. For example, an extension, rotation or a twisting of the flexible body portion may leave the flexible body portion deformed. However, it is desirable for the flexible body portion to readily return to the unconstrained position such that a positioning of the catheter is controllable with precision and reliability. The use of the tensile member comprising the first portion may increase the longitudinal stiffness of the catheter, and accordingly the tensile member may resist elongation of the catheter and/or the flexible body portion when the catheter experiences a tensile force. The tensile member may also aid return of the flexible body portion to the unconstrained position when no force is applied to the catheter. In particular, the tensile member may aid return of the flexible body portion to a rest length when no force is applied to the catheter.

By also reducing a longitudinal stiffness of the tensile member at the second portion such that it is lower than the first portion, the tensile member may still resist elongation and/or return the flexible body portion to an unconstrained position when no force is applied to the catheter, whilst the whipping stress produced by the tensile member is reduced when the tensile member is extended due to a curving of the catheter or otherwise.

It will be appreciated that whilst a longitudinal stiffness of the second portion of the tensile member may be reduced relative to a longitudinal stiffness of the first portion of the tensile member, the longitudinal stiffness of both the first portion and the second portion respectively, when in the unconstrained position, may still be greater than a longitudinal stiffness of the flexible body portion. As such the tensile member may, at all times, increase the longitudinal stiffness of the catheter device.

The second portion being slack along its length when the flexible body portion is in the unconstrained position may effectively reduce the longitudinal stiffness of the tensile member along the second length until the slack has been taken up through flexing of the catheter. The first portion (along the first length) may be considered as taut when the catheter is unconstrained, and the second portion (along the second length) may be considered as loose and/or relaxed when the catheter is unconstrained. Alternatively, the first portion (along the first length) may be also considered as loose and/or relaxed when the catheter is unconstrained, and the second portion (along the second length) may be also loose and/or relaxed when the catheter is unconstrained, but the second portion may comprise a greater amount of slack. That is, the first portion may be elongated by a smaller amount than the second portion, when an equivalent tensile force is applied to both the first portion and the second portion.

For example, the second portion may not be taut until its path length has been extended by a length corresponding to the slack of the second portion. Thus, the longitudinal stiffness of the second portion may be reduced until the second length has been increased beyond a threshold in order to first make the second portion taut. After that threshold the second portion may have the same longitudinal stiffness as the first portion. As the force required to extend the second portion of the tensile member will not substantially increase until the slack has been taken up, i.e. until the second portion is taut, an onset of the whipping stress may be offset such that the likelihood of the whipping effect occurring is reduced.

The second portion may be configured to increase in length at a lower longitudinal stiffness than the first portion when the flexible body portion is curved due to the slack present in the second portion, such that a whipping effect and/or whipping action of the tensile member is avoided. The second portion may therefore extend in length first until it becomes taut. The second portion and the first portion may then increase in combined length with a similar stiffness in terms of longitudinal tension loads.

The tensile member may increase in length when e.g. the catheter is curved. This extension may cause a large amount of energy to be stored in the tensile member, particularly when the tensile member follows the outer radius of curvature. A whipping stress may arise due to the extension, where the tensile member favours a lower energy position such as along the inner radius of curvature. By allowing an extension of the second portion during the curvature before a stretching of the tensile member occurs, the induced whipping stress due to the external applied force on the tensile member may be reduced, and the whipping effect may be avoided. In other words, the longitudinal stiffness of the second portion may be reduced such that the tensile member does not rotate the catheter when the tensile member follows a radius of curvature greater than that of an inner radius of curvature of the flexible body portion when the catheter is actuated.

The tensile member is located at an outer part of the flexible body portion, typically at an outer location in the cross-section of the flexible body portion. The tensile member may extend along a wall of the flexible body portion, and optionally may be within the wall or along an outer surface of the flexible body portion or of a part thereof, such as being between the inner lumen and the outer surface and/or being encased in a suitable outer layer, such as a polymer layer as mentioned elsewhere.

The placement of the tensile member may also alter a torque profile of the catheter. The tensile member may create an asymmetric torque profile of the catheter. The tensile member may be a single tensile member. Altering the torque profile of the catheter may result in desirable responses of the catheter to forces which actuate the catheter, such that various positions of the catheter may be realisable when the catheter is twisted. The altered torque profile may also make it easier to rotate and curve the catheter. This may assist in better placing the catheter, and hence introducing a component to the body, during a procedure.

The tensile member may be located at a single circumferential location relative to the outer part of the flexible body portion. Accordingly the tensile member, which generally increases a longitudinal stiffness of the catheter, may asymmetrically increase the longitudinal stiffness of the catheter about its longitudinal axis. That is, the catheter may be stiffer in a plane where the tensile member is located. The tensile member may also increase a radial stiffness of the catheter. Again, this increase may be located in a plane where the tensile member is located.

Introducing an asymmetry into the torque profile, or the response of the catheter to movement, may allow the catheter to deform to otherwise improbable positions. The tensile member may therefore alter a desired range of movement of the catheter, according to the catheter's intended function.

The flexible body portion may be a tubular member which extends along the length of the catheter. The flexible body portion generally comprises the same shape as the catheter. The flexible body portion may increase a tensile and compression strength of the catheter. The flexible body portion may comprise a tube with a plurality of slits and cuts extending axially and/or circumferentially, which may permit a bending, twisting, angling or curving of the flexible body portion. The flexible body portion may be less stiff in the longitudinal direction compared to other directions.

The flexible body portion may generally define a hollow of the catheter. The surface of the hollow may be defined by the inner surface of the flexible body portion. If the flexible body portion and the tensile member are embedded in polymer, the polymer may define the surface of the hollow. The hollow is generally suitable for the introduction of a component into the body, the component configured to be ejected from the catheter or to remain within the catheter during insertion. The flexible body portion has an outer surface and an inner surface.

The tensile member is in communication with the flexible body portion. That is, the tensile member is configured to exert a force on the flexible body portion when the catheter is not in the unconstrained position.

The tensile member may be in communication directly with the flexible body portion, i.e. by being in contact with the flexible body portion.

The tensile member may be in communication indirectly with the flexible body portion, i.e. through a medium in contact with both the tensile member and the flexible body portion. For example, the tensile member and the flexible body portion may be embedded in a polymer. As such, the polymer serves as a medium for the tensile member to be in communication with the flexible body portion, via indirect contact. The tensile member and the flexible body portion may be regarded as components disposed within a wall of the catheter.

The tensile member may in communication with the flexible body portion via both direct and indirect contact. For example, a distal end of the tensile member may be fixed to a distal end of the flexible body portion, and a proximal end of the tensile member may be fixed to a proximal end of the flexible body portion. The central portion of the tensile member may then be in indirect contact with the flexible body portion via a polymer or other suitable medium.

The catheter generally extends from a proximal end of the catheter to a distal end of the catheter. Distal will generally be understood to be a direction distal from an operator of the catheter, or the direction in which a catheter is to be inserted into the body, i.e. distally. Proximal will generally be understood to be a direction proximal to an operator of the catheter, or in the direction in which a catheter may be retracted from the body, i.e. proximally.

The catheter generally defines a longitudinal axis. Axial will generally be understood to be a direction along the axis of the catheter. Radial will be understood to be in a direction normal to and extending from the axis of the catheter to an outer surface of the catheter. Circumferential will be understood to be in a direction normal to the radial direction and the axial direction, i.e. around a surface of the catheter.

A distal end of the tensile member may be fixed at a distal end of the flexible body portion. A proximal end of the tensile member may be fixed at a proximal end of the flexible body portion.

The catheter will generally be in the unconstrained position when it is in a state of rest, and is not constrained, held or moved by any other body or force. When in the unconstrained position the catheter may generally extend in the axial direction in a straight manner. The unconstrained position may also be considered a resting position. The resting position may be generally straight and in alignment with the longitudinal axis of the catheter. The unconstrained position may also be considered a position at which the catheter has an initial length, or a rest length. Actuation of the catheter may increase a length of the catheter such that its length is extended, or increased, from this initial length. As described above, the tensile member may be configured to aid return of the flexible body portion to an unconstrained position when no force is applied to the catheter. That is, the tensile member may resist an extension of the catheter, and may aid return of the catheter to its initial length.

The catheter may be moved actively or passively. For example, if the catheter is inserted along a vessel, tube or the like which does not extend in a straight manner, the catheter may curve, bend, angle or the like in accordance with the constraints of its container. This may be regarded as passive movement. The catheter may also be moved actively. For example, the catheter may be actuated by a control device either surrounding or in communication with the catheter.

The catheter may be actuated by external forces. For example, the catheter may be housed within a delivery catheter or introducer which, when actuated itself, applies these forces to the catheter. The tensile member may be configured to counteract the external force applied due to the external forces, such that the flexible body portion, and hence the catheter, returns to the unconstrained position when no external force is applied to the catheter.

The catheter may move via extending, bending, curving, twisting or angling. Each of these motions may result in internal actuations of the catheter, and hence change an overall shape of the catheter.

The catheter may also be moved as a whole via rotating or translating the catheter. Each of these motions may move the catheter as a body, and hence do not change an overall shape of the catheter, but change the location of the catheter relative to its environment.

The first portion may extend along a majority of the tensile member. The first portion may be longer than the second portion. The length of the first portion may be greater than the length of the second portion. The first length may hence be greater than the second length. The combined lengths of the first portion and the second portion may equal the entire length of the tensile member.

The second portion may extend along a second length of 20 cm or less, 15 cm or less, or less or 5 cm or less. The second portion may have an axial length between 10 cm and between 11 cm and 19 cm, between 12 cm and 18 cm, between 13 cm and 17 cm, or between 14 cm and 17 cm. The first portion may extend along a first length comprising the remaining length of the tensile member.

The second portion may be located toward a distal end of the tensile member compared to the first portion. The first portion may be located at a proximal end of the tensile member.

The second portion may be located at the distal end of the tensile member and at the distal end of the catheter. Alternatively, there may be a further, third portion of the tensile member at the distal end to provide a short length with the same performance in tension as the first portion. Thus, the second portion may be in the middle of the first portion and the third portion. That is, portions of the tensile member with higher stiffness may be located at a distal end and a proximal end of the tensile member, and the second portion, with lower stiffness due to the slack discussed above, may be located between the first portion(s).

The tensile member may comprise multiple portions corresponding to the first portion (such as the aforementioned third portion). The tensile member may comprise multiple portions corresponding to the second portion (such as a fourth portion). The portions corresponding to the second portion may be located along lengths of the catheter at which a sharp curvature is desirable, such that whipping effects may be avoided due to high degrees of curvature exhibited by the catheter along these regions. Portions corresponding to the first portion and portions corresponding to the second portion (i.e. comprising slack) may alternate along the length of the tensile member. The lengths of each of the portions may also vary in accordance with the desired response of the catheter. As such the catheter may be capable of making multiple sharp bends or curving at extreme angles along multiple locations of its length, whilst resisting elongation due to the provision of the tensile member and avoiding the onset of whipping stresses due to the strategic placement of portions comprising slack when the flexible body portion is in the unconstrained position.

The tensile member may comprise a third portion. The third portion may comprise the same performance in tension as the first portion.

The tensile member may comprise a fourth portion. The fourth portion may comprise the same performance as the second portion in tension and/or as the second portion when the flexible body portion is in the unconstrained position. The longitudinal stiffness of the fourth portion may be reduced relative to the first portion when the flexible body portion is in the unconstrained position. The longitudinal stiffness of the fourth portion may be reduced or increased relative to the longitudinal stiffness of the first portion when the flexible body portion is in the unconstrained position.

A length of the second portion may be understood to be the distance between a proximal end and a distal end of the second portion in the direction of extension along the outer part of the flexible body portion.

The tensile member may be a braided material. The braid may be a flat braid. The braid may be a round or cylindrical braid. The braid may be a tubed braid. The braided material may be any woven, braided material such as braided composite material. The braided material may be Kevlar braid.

The second portion may comprise a loosened portion of braid. The slack may be the result of the loosened portion of braid. The braid may be loosened in a variety of ways. Ultimately, the loosened braid will not be taut. Loosening and/or relaxing the braided material may decrease a total length of the second portion. However, tightening the tensile member when the catheter is curved may increase the length of the second portion, and hence the tensile member.

The loosened portion of braid may comprise a longitudinally compressed portion of the braided material. That is, the braided material may have been axially compressed such that the braided material is loosened. For example, the weaves and/or braid of the material may be motivated in a direction transverse to the longitudinal axis of the tensile member. This may effectively loosen the braid. It will be understood that the loosened braid will comprise slack, as the second portion will need to be pulled in opposite axial directions to tighten the braid.

For example, if the braid is formed generally of two fibres woven together, axially compressing the braid may cause the loosened braid to comprise a helix shape. Tightening the loosened braid may comprise collapsing the helix shape.

The longitudinal compression of the braided material may form a gathering or bunching of the tensile member. For example, if the axial compression of the braid may cause the fibres to increase in density together along the second portion.

The braided material may be a tubular braided material. The loosened portion of braid may comprise a radially compressed portion of the braided material. That is, the braided material may have been flattened from opposing directions such that the braided material is loosened. For example, as the braided material is flattened the weaves and/or braid of the material may be motivated in a direction transverse to the longitudinal axis of the tensile member. This may effectively loosen the braid. It will be understood that the loosened braid will comprise slack, as the second portion will need to be pulled in opposite axial directions to tighten the braid. The braid may comprise folds and/or creases defining the edges of the flattened and/or radially compressed section portion.

The loosened braid may increase a cross-sectional area of the second portion, when the catheter is in the unconstrained position (i.e. the second portion is not taut). The cross-sectional area of the second portion may be greater than the cross-sectional area of the first portion. This may be a result of the loosening of the braid.

For example, axially compressing the braid may symmetrically increase a cross-sectional area of the braid. If the braid is tubular, a radius of the cross-sectional area of the braided material may increase.

For example, if the braid is flat and axially compressed, or if the braid is flattened via radial compression, a major axis of the cross-sectional shape of the braid may increase in length out of a centre of the braid.

The tensile member may deform when extended such that the loosening of the braid of the second portion is reversed. As the tensile member deforms a cross-sectional area of the second portion may correspond to a cross-sectional area of the first portion, as the second portion becomes taut. When this occurs, the second portion may act similarly to the first portion in terms of a response to an extension of the tensile member. Until the slack is tightened, a response of the second portion to the extension of the tensile member may be reduced. Delaying the response of the tensile member to a curvature of the catheter may avoid any whipping effects.

The second portion may comprise a series of undulations extending in a direction perpendicular to a direction in which the tensile member extends. The second portion may comprise a series of undulations extending circumferentially in relation to the flexible body portion. The undulations may extend in a direction parallel to the outer part of the flexible body portion, and transverse to the longitudinal axis of the flexible body portion. The undulations may be shaped in a wave pattern, for example a saw wave, a triangular wave or a sinusoidal wave. The undulations may be in a zigzag shape.

The undulations of the second portion may allow the second portion to longitudinally extend a greater proportion than the first portion when the catheter is curved, such that the tensile member undergoes the same extension for a smaller applied force than if the tensile member solely comprised the first portion. Accordingly a whipping stress may be reduced given the reduced longitudinal stiffness of the second portion relative to the first portion, and a whipping effect may be avoided. The undulations may increase a path length of the tensile member.

The undulations may be regarded as a concertina or bunching of the second portion. The undulations may be regarded as a region of slack. This may apply when the tensile member is formed of braided material or other woven composite materials, as well as when the tensile member is formed of a single wire or non-woven and/or braided material. The undulations may be formed by bending and/or angling the second portion.

The flexible body portion may be a laser cut hypotube. The flexible body portion may be formed of stainless steel or nitinol. The flexible body portion may be formed of a fibre-reinforced polymer material, such as PEEK. The flexible body portion may comprise elastic elements such as metal and/or polymer elements, for example spring type elements. The elastic elements may provide a “backbone” for the catheter, for example an internal shaping and reinforcing structure, which may be embedded in an elastic material, such as an elastic polymer material, along with the tensile member. The flexible body portion may therefore comprise a reinforced polymer backbone, and the catheter may comprise the reinforced polymer backbone and the tensile member surrounded with an elastic polymer material.

As discussed above, the elastic elements may have the form of a tube with cuts permitting bending of the tube, such as an array of slots cut along the length of the tube. One way to form such a tube is by laser cutting of a full tube. The tube may be formed by heat setting a suitable metal, such as nitinol or stainless steel, or other elastic material such as a reinforced polymer material.

The tensile member may be formed of a composite material. The tensile member may be formed of Kevlar. The tensile member may be formed of a braided material. The tensile member may be formed of a polymer or metal material. The tensile member may be formed of a composite braided material. The tensile member may be formed of Kevlar braid.

The tensile member may be cylindrical. The tensile member may be tubular. The tensile member may be flat and/or ribbon-shaped.

The catheter may comprise a liner situated between the backbone for the flexible body portion and the surrounding elastic polymer material. The liner may be a polymer lining, such as Pebax® coated PTFE. The liner may act as a skin, which increases adhesion between the flexible backbone and the surrounding elastic polymer.

The catheter may comprise a composite tube extending along the outer surface of the flexible body portion. The composite tube may be open at each end of the catheter. The composite tube may comprise a lumen. The lumen may be configured for receiving a wire. For example, the wire could be for controlling a component distal to the catheter.

As discussed above, the catheter may comprise an elastic polymer material. The flexible body portion and the tensile member may be embedded in the elastic polymer material. The elastic polymer material may define an outer surface, an inner surface, and openings at each end of the catheter. The elastic polymer material may be a radiopaque material, so that the catheter may be better imaged when inserted into the body.

Viewed from a second aspect of the present invention, there is provided a catheter device for implanting an anchor into body tissue to attach a line to the body tissue, the catheter device comprising: an anchor for implantation in the body tissue to hold a line; and the catheter of the first aspect.

The catheter of the first aspect may be less likely to experience a whipping action or be rotated due to the longitudinal stiffness of the tensile member. Accordingly, undesirable rotation of the catheter within the catheter device may be avoided. This may increase a reliability and/or precision of the catheter device when implanting an anchor in the body tissue to hold a line. Particularly when the anchor is to be implanted in heart tissue, avoiding sudden movements of the catheter device is desirable so that the anchor may be implanted in the correct position, whilst minimising an amount of trauma the body tissue is to experience.

The catheter may be axially offset from a longitudinal axis of the catheter device. The catheter and the catheter device, when the catheter is axially offset from the longitudinal axis of the catheter device, may be configured such that rotation of the catheter device about its longitudinal axis will rotate the catheter when the catheter device is in an unconstrained position.

The longitudinal axis of the catheter device will generally be understood to be the central axis extending along the catheter device, which being a catheter device is of a generally tubular construction. In an unconstrained position, i.e. in a position where the catheter device will generally be in a state of rest, and is not constrained, held or moved by any other body or force, the catheter device may generally extend in the axial direction in a straight manner. The unconstrained position may also be considered a resting position. The resting position may be generally straight and in alignment with the longitudinal axis of the catheter device.

The catheter and the catheter device may generally have a plurality of wires and other components extending through them, for operating components of the catheter device. When the catheter is therefore axially offset from the longitudinal axis of the catheter device, it may be infeasible for the catheter device to rotate without the catheter also rotating. This may be a deliberate construction which ensures smoother operation of the catheter device. This deliberate construction may also result due to the construction of a control handle, which may operate one or more components and/or functions of the catheter and/or the catheter device.

The tensile member of the catheter may be configured to asymmetrically increase a stiffness of the catheter such that when the catheter device is rotated while curved the longitudinal axis of the catheter will align with the longitudinal axis of the catheter device. The catheter device may be configured to rotate independently from the catheter when the longitudinal axes of the catheter device and the catheter are aligned.

The tensile member may be located at a single circumferential location relative to the outer part of the flexible body portion. Additionally, if other composite tubes or the like are present along with the tensile member, they may be located in close proximity to one another rather than being equidistantly spaced from one another around the outer part of the catheter. Accordingly the tensile member, which generally increases a longitudinal stiffness of the catheter, may asymmetrically increase the longitudinal stiffness of the catheter about its longitudinal axis. That is, the catheter may be stiffer in a plane where the tensile member is located. The tensile member may also increase a radial stiffness of the catheter. Again, this increase may be located in a plane where the tensile member is located.

By increasing the stiffness of the catheter asymmetrically, the catheter may have a non-uniform response when moved. This may particularly apply when the catheter is moved actively. Accordingly, the catheter may deflect such that the longitudinal axis of the catheter aligns with the longitudinal axis of the catheter device. This may be due to different tensile forces acting on the catheter dependent on the local stiffness in that part of the catheter.

By providing the tensile member which introduces an asymmetry to the torque profile of the catheter, it may be possible to align the longitudinal axes of the catheter and the catheter device when the catheter and the catheter device are rotated while curved. The catheter device may then be rotated independently from the catheter, such that the relative orientation of the catheter to the catheter device is purposefully altered. This may provide better positioning and functionality for components housed within the catheter and/or the catheter device.

The catheter device may comprise an anchor holder that connects to the anchor whilst it is stowed and during deployment and releases the anchor after successful deployment of the anchor. The anchor holder may comprise a piston for engagement with the anchor and a piston housing for holding the piston, with the piston able to be actuated for sliding movement relative to the piston housing. The piston and the piston housing may be housed within the catheter.

The piston may require a specific orientation for engagement with the anchor. This may be particularly so for ensuring retraction and redeployment of the anchor, if the anchor has not been positioned correctly in the body tissue. Accordingly it is desirable to avoid unintended rotations of the piston. When the piston is housed within the catheter of the first aspect, such unintended rotations due to a whipping action of the catheter may be avoided, and hence the piston may be maintained in a correct orientation for engaging with the anchor.

The piston may include a cutter for cutting of the line once the anchor has been successfully deployed in a desired location, with the line adjusted to a suitable length.

The catheter may comprise one or more lumens, including at least one of: a chordae lumen, i.e. for the line for implantation into the body, and a cutter wire lumen, i.e. for a wire for operating the cutter.

The catheter may be regarded as an adjustment catheter, i.e. a catheter for controlling one or more of the adjustment features of the catheter device such as the adjustment of the line, cutting of the line, or operation of any of the components of the catheter device associated with adjustment of the line.

The catheter device may include a mechanism for control of movement of the piston relative to the piston housing. This may include wires and/or rods of suitable type. These wires and/or rods may be housed in the catheter, optionally by one or more lumens. The mechanism may be arranged to slide the piston outward from the piston housing in order to move the anchor away from the piston housing. The mechanism may be further arranged to draw the piston back into the piston housing to either disengage the piston from the anchor or to draw the anchor back toward the piston housing along with the piston. However, if it is determined that the anchor is not placed correctly then the user may decide to draw the anchor back toward the piston housing in order to then pull the anchor along with the adjustment housing back into the distal part to withdraw the anchor from the body tissue and fold the hooks back into the folded, stowed position.

To prevent the cutter from exceeding its desired range of motion, the cutter may be equipped with two stopping features disposed at an upper and lower end of the cutter. To prevent the cutter from moving further than its upper position in the piston housing, a cutter wire may be threaded through the housing and/or the cutter to stop the cutter in an upper position. Even if the cutter wire were to break, the cutter and a wire attached to the cutter operating it cannot escape from an upwards end of the housing as both are contained within the housing. To prevent the cutter from moving further than its lower position in the housing, a cam or the internal cam may function as the lower position stopping feature.

The catheter device may comprise a housing section extending from a distal end of the catheter device along the length of the catheter device toward the proximal end of the catheter device, the housing section comprising a distal part at the distal end of the catheter device and a proximal part located on the proximal side of the distal part. The catheter device may comprise an anchor deployment mechanism at the distal part of the housing section for deployment of the anchor for attachment of the anchor to the body tissue, wherein the anchor deployment mechanism is arranged for deployment of the anchor from a stowed position of the anchor by moving it outward in the distal direction relative to the distal part. The anchor may be held in its stowed position by the anchor deployment mechanism in the distal part prior to deployment.

The anchor deployment mechanism may comprise an adjustment housing that holds the anchor during deployment and facilitates adjustment of the line. The adjustment housing may form the anchor holder. The adjustment housing may also be housed within the catheter.

The anchor may comprise a number of hooks for engagement with the body tissue and having a folded position and an unfolded position, wherein the anchor is made of an elastic material such that the hooks can be elastically deformed into the folded position by application of a constraining force, and will return to the unfolded position when no constraining force is applied, and wherein the hooks are held in the folded position whilst the anchor is in the stowed position within the distal part.

The anchor may include a locking mechanism with an elastically deformable locking segment. The locking mechanism may be for locking the line in place after deployment of the anchor. The anchor deployment mechanism may be arranged to hold the locking segment in a deformed position when the anchor is stowed within the distal part. In one example the locking segment is tubular when it is not deformed, and may align with a tubular wall of the anchor, with deformation of the locking segment moving it out of alignment with the tubular wall of the anchor. The anchor may have a circular tubular wall, with the deformed locking segment having a non-circular form with an ovoid shape where parts of the locking segment protrude outward beyond the tubular walls of the anchor.

The piston may include a piston wedge for engagement with a deformable element of the anchor, which advantageously is a locking segment as mentioned above. The piston wedge may be a wedge shaped section at the distal end of the piston. The wedge-shaped section advantageously assists in engaging the locking segment and equally disengaging the locking segment due to its shape. The piston wedge may be arranged to be pushed between the locking segment and the wall of the anchor to elastically deform the locking segment, advantageously forming the non-circular form of the locking segment as well as opening the locking segment to allow for adjustment of the line. In that case, when the anchor is in the stowed position the piston wedge is engaged with the locking segment. The piston wedge may be a two-legged fork with an opening allowing for the line to pass between the two legs (tines) of the fork.

The piston wedge may be engaged with the locking segment without being in contact with any other wall of the papillary anchor. Thus, the anchor holder and the anchor may be arranged such that when the piston wedge is engaged then it is spaced apart from the wall of the anchor. By advantageously requiring that the piston wedge is in contact with the locking segment of the anchor alone, and not any other wall of the anchor, the piston wedge experiences less friction from the anchor. As such, during deployment of the anchor from the catheter device, the anchor may deploy without the piston wedge moving with the anchor to thus ensuring the locking of the locking segment. The placement of the piston in the anchor holder acts as a cantilever, which prevents the piston wedge from being pulled towards the wall of the anchor due to the elastic force of the locking segment. The piston and hence the piston wedge may be made of a suitably rigid material, such that the piston wedge is not bent out of shape by the reaction force due to the cantilever action of the piston and the force exerted on the piston wedge by the locking segment acting in opposite directions.

The piston may include the cutter, with this cutter being arranged to cut the line when the piston is withdrawn from the anchor. The cutter on the piston may be a cutting surface arranged to interact with a surface of the piston housing to cut the line, such as via a shearing action. Thus, where a piston wedge is used then the withdrawal of the piston may allow the line to be locked in place at the anchor as the locking segment returns to its undeformed position and clamps the line to the anchor wall, whilst the line is simultaneously cut by the cutter. In this way the piston aids in an adjustment and cutting procedure once the anchor is correctly placed and the length of the line is as required.

An internal cam may be provided for aiding in holding the anchor locking segment in an open position. The internal cam may have an unexpanded configuration where the cam fits inside the locking segment in the undeformed state of the locking segment, and an expanded configuration where the cam fits inside the locking segment in the deformed state, i.e. the non-circular form discussed above. The cam may have an opening at its centre that is wider in the expanded configuration than in the unexpanded configuration. The piston may be provided with a cam wedge for urging the opening of the cam to the wider state and hence expanding the cam. The cam wedge may be provided in addition to the piston wedge discussed above, so that a single piston has a fork like form at its distal end, with at least one tine of the fork providing the cam wedge and at least one tine of the fork providing the piston wedge. The cam may be arranged aid opening of locking segment and it may also act to fix the anchor to the adjustment catheter, where present. The cam may be held in place by a cam holder on the adjustment housing.

In some examples, the anchor is provided with a locking mechanism that clamps the chord when no force is applied, and that can be elastically deformed to release the chord for adjustment of the length of the chord during implantation thereof. As noted above, the locking mechanism may comprise a resiliently deformable locking segment. The locking segment may be formed in a wall of the anchor and divided from the wall by one or more slit(s). The anchor may be arranged so that when no forced is applied then the slits are closed with no gap or a relatively narrow gap in order to clamp the line, whereas when a suitable force is applied to the locking segment and/or wall then the locking segment and/or the wall will elastically deform to widen the opening provided by the slit(s) so that the line is released. The anchor may have a tubular body section, in which case the locking segment may be formed in the wall of the tube. The locking segment may be a band with parallel slits on two sides, such that the band can be pulled out of plane with the wall by application of a force in order to open up the slits. Advantageously, this movement of the locking segment may create the non-circular form for the anchor. Such a locking segment can be held open by sliding a holder into the slit(s), such as the piston discussed above.

The housing section may be formed from one or more tubular sections in any suitable material, i.e. a medically appropriate material. Stainless steel or nitinol may be used. Polymeric materials are also an option. In the alternative, composite materials such as carbon-fibre or glass-fibre reinforced PEEK may be used. The catheter device may be formed via a combination of such materials with the materials for different parts of the device being selected dependent on the required characteristics of those parts. A material that allows ultrasound to pass through and at the same time have sufficient strength is preferred, carbon reinforced PEEK meets these demands well, and would also allow injection moulding of the components which lowers manufacturing cost. Fibre reinforced plastics are normally not visible on X-ray, so strategically placed radiopaque markers in all components may be used to determine device component(s) position and orientation on X-ray relative to each other, as complementary information to ultrasound imaging.

As mentioned above the catheter device may be for implanting the anchor into the heart and the anchor may be a papillary anchor for implantation into the papillary muscle, with the line for example being an artificial chordae line. The catheter device may also be arranged for implanting a leaflet anchor along with the papillary anchor into the heart as part of a procedure for implanting an artificial chordae line that extends between the leaflet anchor and the papillary anchor. Thus, the catheter device may further include the leaflet anchor and a leaflet anchor deployment mechanism.

Thus, the housing section may be a two-part housing section, the two-part housing section being arranged to be placed between the papillary muscle and a leaflet of the heart during use of the catheter device, and the two-part housing section comprising a distal part at the distal end of the catheter device and a proximal part located on the proximal side of the distal part; wherein the distal part holds the papillary anchor deployment mechanism, i.e. the anchor and adjustment housing/anchor holder as discussed above, and the proximal part holds the leaflet anchor deployment mechanism.

The leaflet anchor and/or the leaflet anchor deployment mechanism may be similar to that of WO 2020/109599.

An example of the use of the catheter device may include the following steps: (1) the device is first placed in near proximity to final placement; (2) the distal part is moved toward the body tissue that is to receive the anchor; (3) the distal end of the distal part meets the body tissue, and as force is applied the counterforce from the body tissue eventually surpasses the forces holding the anchor in place, at this point tissue is pushed flat below the base of the device giving a maximal chance of placing all hooks of the anchor correctly in tissue, and force can be applied to the anchor so that the ends of the hooks then move beyond the distal end of the distal part to meet the body tissue, this may be done via additional force on the anchor and/or the anchor deployment mechanism from rods or wires, or advantageously it may be done through a pre-tension on the anchor that is held by friction with the distal part until the forces from the body tissue on the distal part changes the balance of forces with the friction sufficiently so that the anchor ejects (similar to a paper stapler); (4) the anchor hooks fold out and form into the hook shape of the unconstrained anchor to thereby engage with the body tissue, at which point the connection can be pull tested by operator, and/or visually confirmed on x-ray and/or ultrasound; (5) if the connection is not satisfactory, the anchor can be pulled back into the device and re-placed to attempt an improved coupling of the anchor with the body tissue.

Viewed from a third aspect of the present invention, there is provided a steerable introducer for a catheter device, the steerable introducer comprising: a body portion having a longitudinal axis extending from a proximal end of the introducer toward a distal end of the introducer, wherein the body portion comprises a flexible distal portion extending to the distal end; a hollow within a part of the body portion holding the catheter of the first aspect; and at least one steering control wire extending axially along the body portion and along at least a part of an axial length of the flexible distal portion; wherein the at least one steering control wire has a distal end that is fixed to the flexible distal portion at a first point in a first axial and circumferential position on the flexible distal portion, with the introducer being arranged to allow for tension on the at least one steering control wire to cause a curvature of the flexible distal portion; and wherein the catheter is configured to follow the curvature of the flexible distal portion.

The steerable introducer may comprise a pair of twisting control wires extending axially along the body portion and along at least a part of an axial length of the flexible distal portion. The pair of twisting control wires may each have a distal end, with the twisting control wire distal ends being fixed to the flexible distal portion at second and third points at respective second and third axial and circumferential positions on the flexible distal portion, with the circumferential position of the twisting control wire distal ends being different from each other as well as different from the circumferential position of the steering control wire, and with the introducer being arranged to allow for variations in relative tension between the pair of twisting control wires to cause twisting of the flexible distal portion.

Thus, with this arrangement the introducer is both steerable and also twistable, such as to create an angling of the distal end whilst the flexible distal portion is curved and optionally a side-to-side panning motion with a consistent angle of the distal end. This can allow for the introducer to fit with complex anatomy and to approach a target site through a convoluted route, which still being able to adequately target the deployment of the catheter device, for example in the case of repair within the mitral space as with the catheter device of WO2016/042022, using just two shafts, being the introducer shaft and separately the shaft of the catheter device. It is important to realise that repair devices for the mitral space generally consist of three shafts, with an introducer, a quad (steerable) shaft and a device shaft. It is beneficial from many perspectives to have as few shafts as possible (e.g. ease of production, ease of use, and reduced risk of failure including both device failure and decreased risk to the patient). The steerable introducer of the first aspect can allow for repairs in the mitral space using only two shafts.

The flexible distal portion can be subject to a curvature within a plane under the influence of the steering control wire, which can be paired with a rotation of the introducer in the manner described above to steer it to a target site, such as by navigation through blood vessels. In addition, the ability to also apply twisting forces via the pair of twisting control wires allows for a finer control of the location and orientation of the distal end of the introducer, and hence of the location of the catheter device as it is deployed. Advantageously, the distal end of the introducer can be moved via the twisting movement simultaneously with the steering movement, so that the distal end may shift out-of-plane from the plane of the curvature that would be the result of tension on the steering control wire alone. With only a steering input the distal flexible portion of the introducer would curve in a plane, whereas with a steering and a twisting input the curvature defines a more complex shape along a three dimensional surface. An added twisting movement can have significant advantages when the correct placement of the distal end is of importance for correct targeting of the catheter device, such as for ensuring correct targeting of the mitral valve for the device of WO2016/042022.

The curvature and/or twisting movement of the steerable introducer may be imparted on the catheter held within the hollow. The catheter may hence be constrained by the steerable introducer.

By requiring that the catheter comprises the tensile member, rotation of the catheter due to whipping stresses when constrained by the steerable introducer may be avoided. The effects of this sudden rotation, such as moving or destabilising the steerable introducer, may equally be avoided. Therefore the steerable introducer may be able to curve without becoming destabilised by adverse rotation of the catheter, which may also avoid further trauma in the body tissue surrounding the steerable introducer.

Viewed from a fourth aspect of the present invention, there is provided a steerable introducer for a catheter device, the steerable introducer comprising: a body portion having a longitudinal axis extending from a proximal end of the introducer toward a distal end of the introducer, wherein the body portion comprises a flexible distal portion extending to the distal end; a hollow within a part of the body portion holding the catheter device of the second aspect; at least one steering control wire extending axially along the body portion and along at least a part of an axial length of the flexible distal portion; and a pair of twisting control wires extending axially along the body portion and along at least a part of an axial length of the flexible distal portion; wherein the at least one steering control wire has a distal end that is fixed to the flexible distal portion at a first point in a first axial and circumferential position on the flexible distal portion, with the introducer being arranged to allow for tension on the at least one steering control wire to cause a curvature of the flexible distal portion; and wherein the pair of twisting control wires each have a distal end, with the twisting control wire distal ends being fixed to the flexible distal portion at second and third points at respective second and third axial and circumferential positions on the flexible distal portion, with the circumferential position of the twisting control wire distal ends being different from each other as well as different from the circumferential position of the steering control wire, and with the introducer being arranged to allow for variations in relative tension between the pair of twisting control wires to cause twisting of the flexible distal portion.

Viewed from a fifth aspect of the present invention, there is provided a method of use of a catheter for implanting an anchor into body tissue for implanting an artificial chordae line, the method comprising: inserting the catheter into the body. The catheter may be the catheter of the first aspect and optionally may include other features thereof as discussed above. The catheter may be part of the catheter device of the second aspect and optionally may include other features thereof as discussed above. The method may include positioning a distal end of the catheter in the body tissue. The positioning may include, for example, bending, twisting, angling and/or curving the catheter.

Viewed from a sixth aspect of the present invention, there is provided a method of manufacture of the catheter of the first aspect, the method comprising: embedding the tensile member and the flexible body portion in a soft polymer.

The soft polymer may be Pebax. The tensile member and the flexible body portion may be embedded in the soft polymer using Pebax reflow. The use of Pebax may allow for sufficient flexure of the catheter.

It is considered to offer particular benefits to be able to form the catheter of the first aspect using the method of the sixth aspect, although it should be noted that other manufacturing methods may be used. The method of the sixth aspect may include providing the catheter with any of the features discussed above with reference to the various device aspects.

Certain example embodiments of the invention will now be described by way of example only and with reference to the accompanying drawings in which:

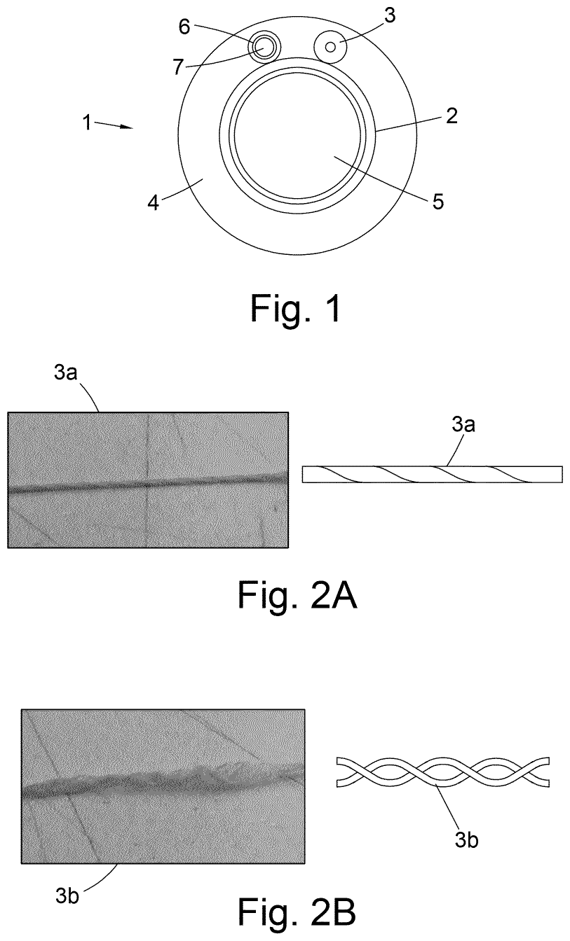

FIG. 1 shows a cross-sectional view of a catheter;

FIG. 2A shows a tensile member in a taut configuration;

FIG. 2B shows a tensile member in a loosened configuration;

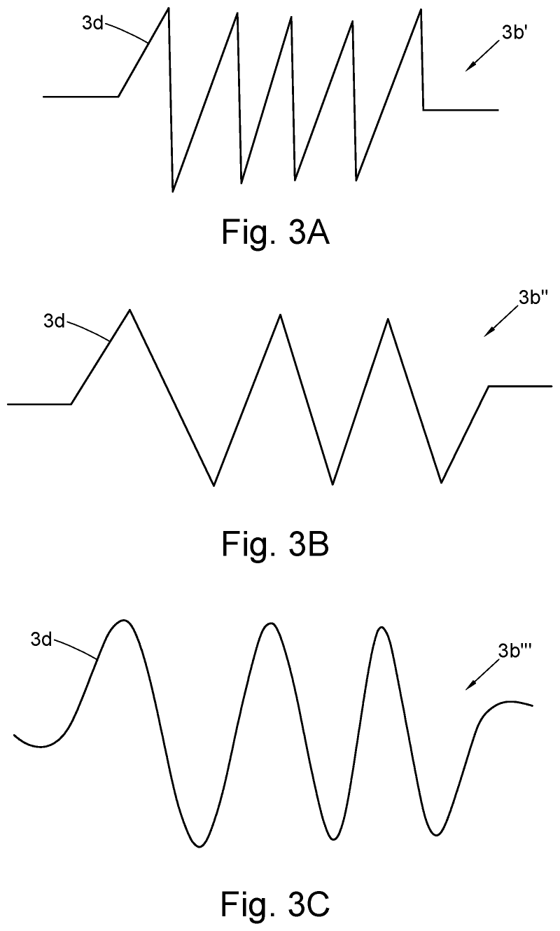

FIGS. 3A to 3C show alternative configurations of a second portion of a tensile member;

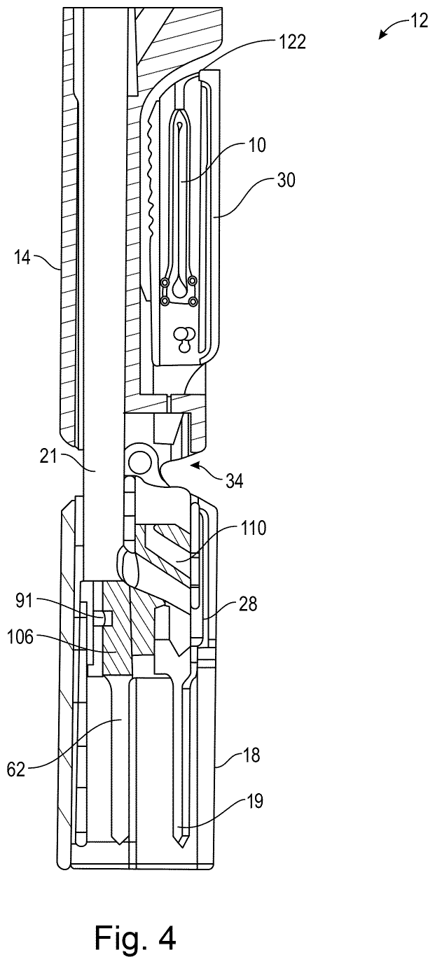

FIG. 4 shows a cross-sectional view of a catheter device;

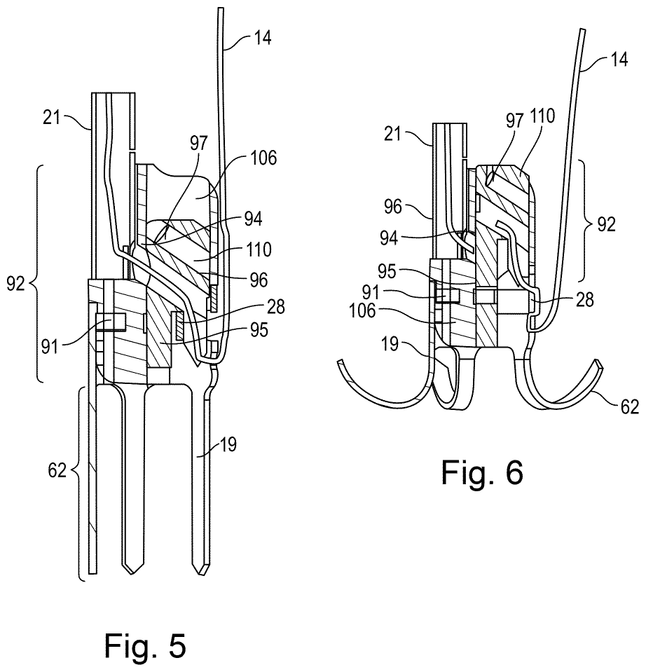

FIG. 5 shows a cross-sectional view of a papillary anchor in an undeployed, unfolded state;

FIG. 6 shows a cross-sectional view of the papillary anchor in a deployed, unfolded state;

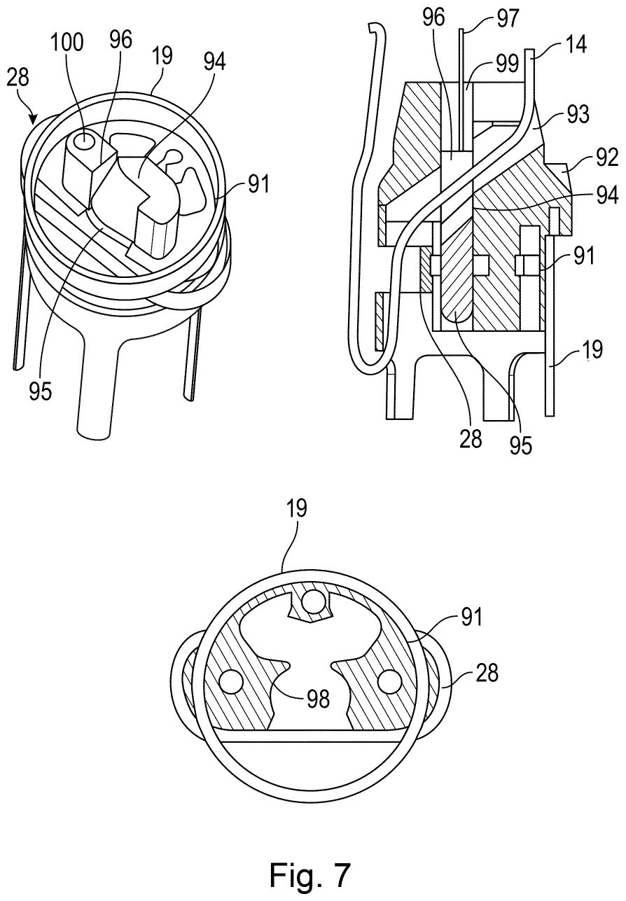

FIG. 7 shows an alternative way of arranging the adjustment and cutting features for the papillary anchor of the catheter device;

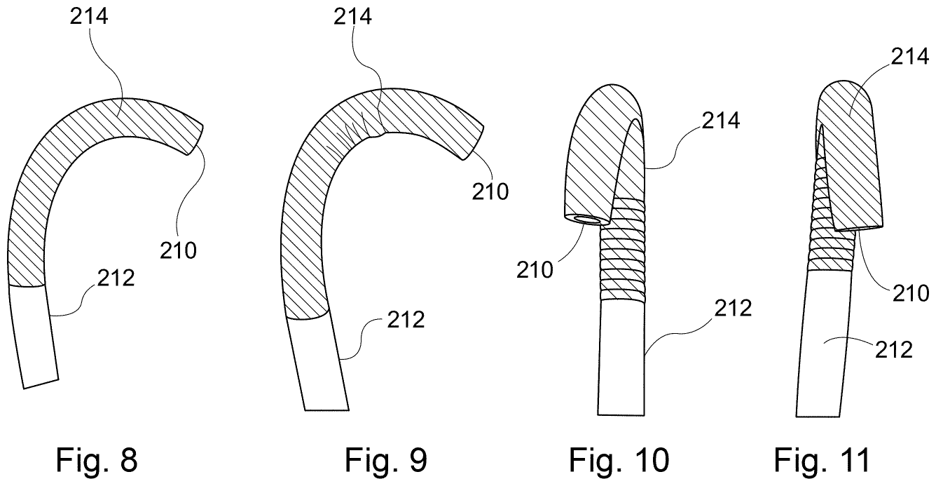

FIGS. 8 to 11 show various views of a flexible distal portion of a steerable introducer with different curvature and twist at the distal end;

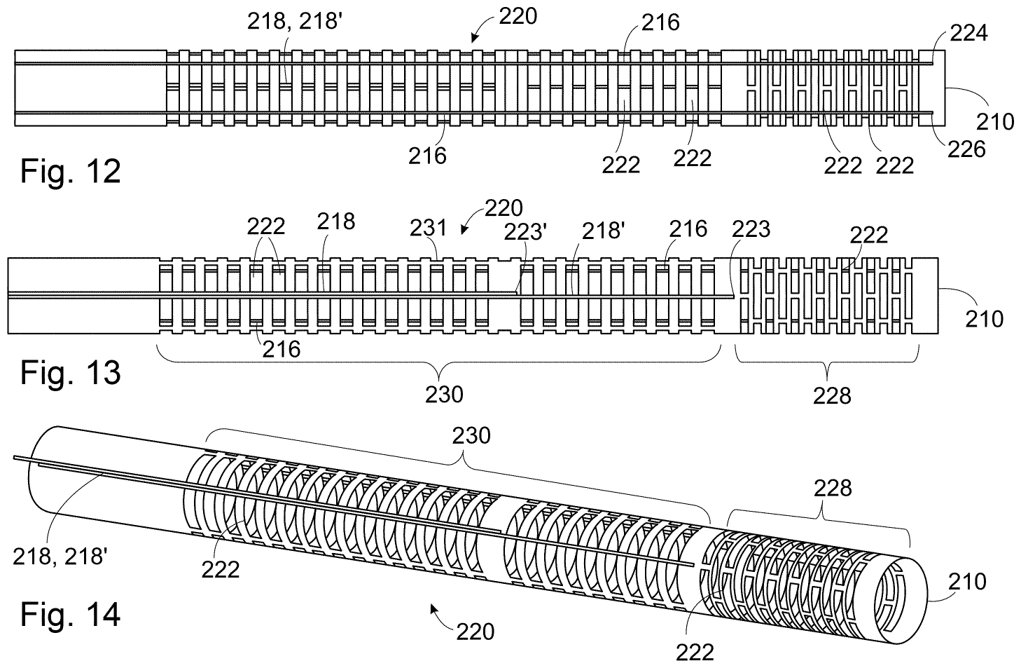

FIGS. 12 to 14 are two side views and a perspective view of an example configuration for an elastic element of the flexible distal portion and the positions of control wires;

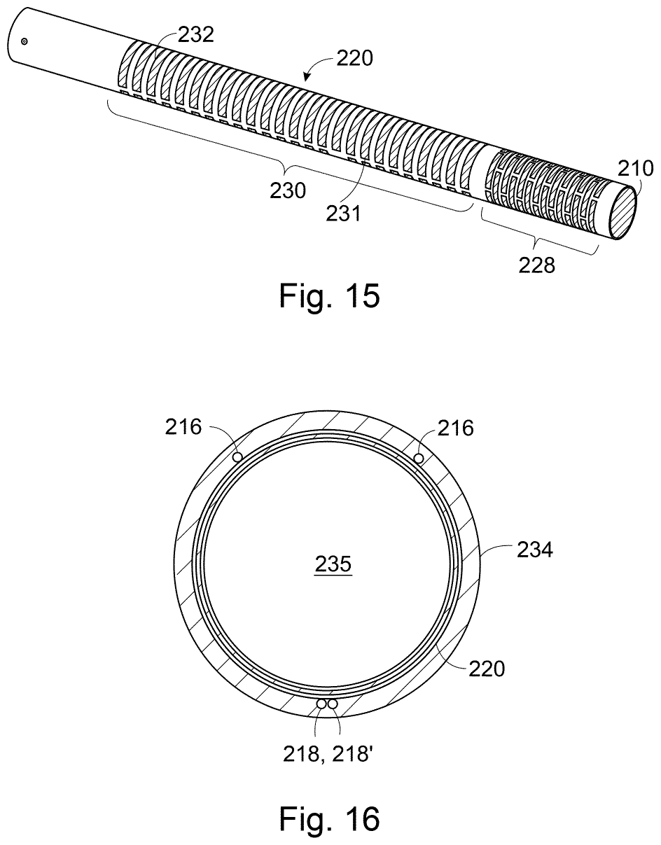

FIG. 15 shows the use of an elastic material in openings of the elastic element;

FIG. 16 is a cross-section of an example in which the elastic element is embedded in an elastic material;

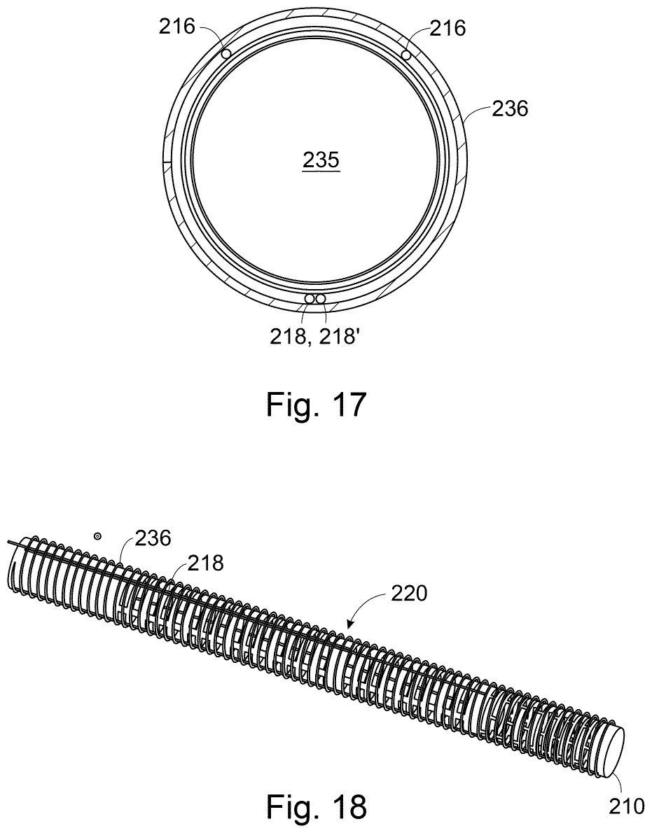

FIG. 17 shows a similar cross-section with the elastic material omitted to allow a coil to be seen; and

FIG. 18 is a perspective view of the same components as FIG. 17;

FIG. 1 shows a cross-sectional view of a catheter 1. The catheter 1 comprises a flexible body portion 2 and a tensile member 3 embedded in polymer 4. The catheter 1 comprises a hollow or lumen 5, and further comprises a composite tube 6 also embedded in the polymer 6. The polymer 6 generally defines the outer surfaces of the catheter 1, which is cylindrical or tubular in shape. The flexible body portion 2, the tensile member 3 and the composite tube 6 all extend along the length of catheter 1. The catheter 1 has a longitudinal axis extending along it, from one opening of the lumen 5 to an opposing opening of the lumen 5.

At rest the catheter 1 extends in a generally straight direction along the longitudinal axis such that the openings defined at either end of the lumen 5 of the catheter 1 are parallel. This position of the catheter 1 may be regarded as an unconstrained position, i.e. a position the catheter 1 will assume when no force is applied to the catheter 1. At rest the catheter 1 will also have a rest length.

During use, e.g. when inserted into the body, the catheter 1 can be moved by translation or rotation, which will move the catheter 1 as a whole, or globally. The catheter 1 is also flexible, so that the catheter 1 can be moved locally such that the catheter 1 may curve or twist along its length.

The flexible body portion 2 may not be capable of extending via an elastic process, and as such may not return to its initial length when it experiences tensile forces. As such a catheter not comprising the tensile member 3 may elongate or extend when a tensile load is applied, and may not return to its rest length when the tensile load is removed.

The catheter 1 may move actively or passively. That is, the catheter 1 may flex such that it conforms to the shape of an object which constrains it, such as when the catheter 1 is inserted into an artery or vein which is not straight, or may be flexed actively by an external force, such as by a steerable introducer which constrains the catheter 1.

The tensile member 3 increases a longitudinal stiffness of the catheter 1 such that the catheter 1 may resist any elongation, or such that any extension of the catheter 1 is an elastic process. The tensile member 3 has a longitudinal stiffness greater than that of the flexible body portion 2, which may generally not resist elongation or extension due to tensile forces. The tensile member 3 reduces elongation of the catheter 1 when the catheter 1 experiences a tensile force, or load acting longitudinally on the catheter 1. The tensile member 3 may also be considered to aid return of the catheter 1 to its unconstrained position, by restoring the catheter 1 to its rest length following any movement which may have extended the catheter 1.

The tensile member 3 comprises a first portion 3a extending along a first portion or length of the tensile member 3, and a second portion 3b extending along a second portion or length of the tensile member 3. The second portion 3b is slack along its length when the catheter 1 is in the unconstrained position. That is, the second portion 3b generally comprises a region of slack where the tensile member 3 is not held taut and/or is loosened.

By providing a tensile member 3 which comprises a loosened and/or slack portion, i.e. the second portion 3b, the tensile member 3 may still increase the longitudinal stiffness of the catheter 1 such that it resists extension under significant loads and returns to its rest length, but may avoid whipping effects experienced by catheters comprising tensile members which do not comprise a loosened and/or slack portion. This is because the longitudinal stiffness of the tensile member 3 may be effectively reduced until the slack is taken up from the second portion 3b by tightening the tensile member 3, which occurs as the tensile member 3 is extended or experiences tensile forces. As such, undesirable rotation of the catheter 1 caused by a whipping stress induced by the tensile member 3 can be avoided.

FIGS. 2a and 2b illustrate the first portion 3a and the second portion 3b of the tensile member respectively. It will be understood than when taut or tightened, the second portion 3b will look and behave similarly to the first portion 3a.

The tensile member 3 illustrated in FIGS. 2a and 2b is a braided or woven material, and generally extends continuously in a direction corresponding to the lengthwise, or longitudinal, direction of the catheter 1. The tensile member 3 comprises a plurality of fibres, wires or strands which are woven or braided to form the tensile member 3. The tensile member 3 may be formed of a composite material, such as Kevlar.

As shown in FIG. 2A, the first portion 3a of the tensile member 3 generally comprises little to no slack. The fibres and/or strands forming the first portion 3a are compact, such that the first portion 3a will be generally taut under little to no tensile force.

As shown in FIG. 2B, the second portion 3b of the tensile member 3 comprises slack along its length. The braided material has been axially compressed, such that the fibres and/or strands forming the second portion 3b have bunched together. The fibres are splayed in a direction perpendicular to the direction of extension of the tensile member 3, and as such the second portion 3b is loosened when the catheter 1 is unconstrained.

In the second portion 3b, a path length of the fibres from a first end to a second end of the second portion 3b will be greater than a displacement between the first end to the second end of the second portion 3b. In comparison, the path length of the fibres from the first end to the second end of the first portion 3a will be generally equal to the displacement between the first end and the second end of the first portion 3a for the first portion 3a, or for the second portion 3b when the second portion 3b is taut.

Due to the introduced slack into the second portion 3b, the longitudinal stiffness of the tensile member 3 will be reduced until the slack is taken up through flexing of the catheter 1. As the longitudinal stiffness of the tensile member 3 is reduced up until a certain amount of extension of the catheter 1, the tensile force required to extend the catheter 1 a set amount will be reduced compared to a catheter 1 which did not comprise a tensile member 3 comprising a first portion 3a and a second portion 3b. As such, when the catheter 1 is curved by significant amounts such as 180° the onset of whipping stresses which may rotate the catheter 1 can be avoided. However, as the tensile member 3 still increases the longitudinal stiffness of the catheter 1, the catheter 1 will still resist elongation due to tensile forces, and will return to its rest length when unconstrained.

The catheter 1 extends from a proximal end, i.e. where an operator of the catheter may be located, to a distal end of the catheter, i.e. at an end which is first inserted into the body

The catheter 1 will generally undergo significant or the most curving at a distal end of the catheter 1. The distal end of the catheter 1 may also generally be where components or devices are located and/or deployed from during a procedure on the body, and as such more sudden changes of direction may be expected in the distal end as the catheter 1 is correctly positioned within the body.

As the most significant flexing will occur at the distal end of the catheter 1, the second portion 3b may be located at or towards the distal end of the catheter 1, whilst the first portion 3a will be located at the proximal end of the catheter 1.

The second portion 3b may extend a distance of around 15 cm or less along the tensile member 3. Such distances are found to prevent the onset of whipping stresses, whilst still providing enough longitudinal stiffness to the catheter 1 such that any undesirable extension of the catheter 1 may be prevented.

FIGS. 3A-C show alternative ways of introducing slack into the second portion 3b′, 3b″, 3b′″ of the tensile member. In each of FIGS. 3A-C, the slack comprises one or more undulations 3d extending in a direction perpendicular to a general direction in which the tensile member 3 extends. The direction in which the undulations 3d extend may be in a circumferential direction of the flexible body portion 2 and the catheter 1. The undulations 3d extend in a first direction perpendicular to the direction in which the tensile member 3 extends, before extending in a second direction opposite to the first direction. The second direction is also perpendicular to the direction in which the tensile member extends. The undulations 3d may be regarded as being shaped in a wave pattern.

FIG. 3A shows the second portion 3b′ of the tensile member 3 comprising a series of undulations 3d in a saw-tooth pattern, or saw wave. FIG. 3B shows the second portion 3b″ of the tensile member 3 comprising a series of undulations 3d in a triangular wave, or a zigzag formation. FIG. 3C shows the second portion 3b″ of the tensile member 3 comprising a series of undulations 3d in a sinusoidal wave.

Similarly to the loosened braid of the second portion 3b in FIG. 2B, the undulations 3d introduce slack into the second portion 3b′, 3b″, 3b″ by increasing the path length, or distance covered by the second portion 3b′, 3b″, 3b″ relative to a displacement between a first end and a second end of the second portion 3b′, 3b″, 3b″. The second portion 3b′, 3b″, 3b″ will only be taut once the slack has been taken up, and until this point the effective longitudinal stiffness of the tensile member 3 will be reduced compared to when the tensile member 3 is completely taut.

Referring back to FIG. 1, the tensile member 3 is shown located in a single plane of the circumference of the catheter 1. As such the tensile member 3 asymmetrically alters the longitudinal stiffness of the catheter 1. This may alter a torque profile of the catheter 1, such that when the catheter 1 is twisted the catheter 1 may be constrained into positions it could not otherwise be constrained to. The asymmetric longitudinal stiffness of the catheter 1 can be used to get varying alignments between the catheter 1 and external devices which may constrain the catheter.

The flexible body portion 2 is formed of an elastic material, and may be formed of stainless steel, nitinol or other suitable elastic materials. The flexible body portion 2 may be a laser cut hypotube, and may also comprise a series of cuts and/or slits along its length, extending circumferentially, which aid the flexible body portion 2 in flexing, such as by either bending, curving or twisting. Whilst the slits may reduce a longitudinal stiffness of the flexible body portion 2, such that it may not return to a rest length when extended or when a tensile force is applied to the flexible body portion 2, the tensile member 3 aids return of the flexible body portion 2 to the rest length by increasing the longitudinal stiffness of the catheter 1, including the flexible body portion 2.

The flexible body portion 2 is in communication with the tensile member 3 by being in contact with the tensile member 3, and also by both the flexible body portion 2 and the tensile member 3 being embedded in the polymer 4. The polymer 4 acts as a medium which transfers any forces or loads across the entirety of the catheter 1.

The catheter 1 shown in FIG. 1 is also provided with a composite tube 6. The composite tube is open at each end of the catheter 1 and defines a lumen 7. The lumen 7 can accommodate components such as wires or cords. The composite tube 6 may provide protection to any wires or cords located within the lumen as the catheter 1 is flexed, and may prevent the cords or wires from becoming tangled with any components located within the lumen 5 of the catheter 1.

FIG. 4 shows a cross-sectional view of a catheter device 12. The catheter device 12 is substantially similar to that as disclosed in WO 2020/109599. The catheter device 12 comprises a gripper housing 4 and a papillary anchor housing 8, which form a proximal part 4 and a distal part 8 of a two-part housing section of the catheter 1. Between the proximal part 4 and the distal part 8 is a flexible joint 34. The proximal part 4 includes a gripper arm 30 shown engaged with the gripper housing 4 in a closed position, ready to deploy a leaflet anchor 10. The papillary anchor housing 8 includes a papillary anchor 19, ready to be deployed into the papillary muscle of the heart.

Running through the catheter device 12 of FIG. 4 and into a distal end of the catheter device 12 is an adjustment catheter 21. As described above, the adjustment catheter 21 is able to control the extension of the flexible joint 34 by means of wires and/or rods 60. The same wires and/or rods 60 also push out the papillary anchor 19 for deployment. As described above, the papillary anchor 9 comprises a number of pins 62 that in an unconstrained configuration form a number of hooks, and further comprises a locking segment 28 disposed within a wall of the papillary anchor 19. The papillary anchor 19 is housed within the papillary anchor housing 18. Further housed within the papillary anchor housing 18 is an adjustment housing 92. The adjustment housing comprises a piston 110, an anchor holder 106 and a cam 91. To prevent unwanted deployment of the papillary anchor 19, a deployment lock mechanism (not shown) using a latch may be disposed within the papillary anchor housing 18. The deployment lock mechanism is actuated via a locking spring that acts on the latch and a deployment lock wire. The deployment lock wire may be situated within the adjustment catheter 21 for operating the latch.

FIG. 5 illustrates a cross-sectional view of the papillary anchor 19 when undeployed with the adjustment housing 92 mounted on top. Contained within the adjustment housing 92 is an anchor holder 106, a piston 110 and a cam 91. The piston 110 comprises a fork-wedge formation 95, which is configured to elastically deform the cam 91 and the papillary anchor 19, and a cutting wedge 96, which is configured to cut the chordae 14 in combination with a cutting section 94 of the anchor holder 106. The fork-wedge can be considered with two main parts, a cam wedge where at least one tine of the fork-wedge 95 is used to open the cam 91, and a piston wedge where at least one tine of the fork-wedge 95 is used to open the locking segment 28. The pointed end of the piston wedge advantageously assists in deflecting the locking segment 28 when the piston wedge and the locking segment are engaged, making engagement/deployment of the anchor 19 with the piston wedge easier. When the cam 91 is engaged by the fork-wedge 95, the cam 91 elastically deforms the locking mechanism 28 of the papillary anchor 19 to an open position. In the configuration shown in FIG. 37, the open locking mechanism 28 and the positioning of the piston 110 within the adjustment housing 92 allows the chordae 14 to slide through with minimal friction. The chordae 14 is attached to the leaflet anchor 10 (not shown) above the papillary anchor 9. In this configuration the chordae is thus easily adjustable in length. The piston 110 ideally features a piston wire location 97 which allows a pull-wire (not shown) to be attached to the piston 110. The pull-wire is ideally disposed through the adjustment catheter 21 (inside a separate lumen in the adjustment catheter 21) without running through the path proximal to the cutting wedge 96 and cutting section 94. When the piston pull-wire is pulled, the piston 110 slides in a direction away from the papillary anchor 19.

FIG. 6 illustrates a cross-sectional view of the papillary anchor 19 when deployed with the adjustment housing 92 mounted on top. The piston 110 slidably moves away from the papillary anchor 19 during deployment. In doing so the fork-wedge 95 of the piston is no longer engaged with the cam 91 and the locking segment 28. The cam 91 no longer elastically deforms and the cam 91 as well as the locking segment 28 of the papillary anchor 19 return to their at rest/undeformed positions. In doing so, the chordae 14 is locked in position and its length is no longer adjustable. Concurrently, when the locking mechanism 28 returns to its undeformed position the cutting section 94 and the cutting wedge 96 cut the chordae 14. Thus in one motion the papillary anchor 19 may be deployed and the chordae 14 suitably attached in place. By disposing the piston pull-wire in the piston pull-wire location 97 above the cutting location, the piston pull-wire may avoid being cut in the same action and thus leaves the device fully operational should readjustment be required.

To prevent the piston 110 from exceeding its desired range of motion, the piston 110 may be equipped with two stopping features disposed at an upper and lower end of the piston 110. To prevent the piston 110 from moving further than its upper position in the housing 92, a cutter wire (not shown) may be threaded through the housing and/or the piston to stop the piston 110 in an upper position. Even if the cutter wire were to break, the piston 110 and a wire attached to the piston 110 operating it cannot escape from an upwards end of the housing 92 as both are contained within the housing 92. To prevent the piston 110 from moving further than its lower position in the housing 92 is the cam 91.

The catheter 1 can be used as the adjustment catheter 21 of the catheter device. Thus, during introduction of the catheter device 12 into the body, the adjustment catheter 21 can comprise a tensile member 3 including a first portion 3a and a second portion 3b, 3b′, 3b″, 3b″ such that the onset of whipping stresses induced due to an extension of the tensile member 3 when the adjustment catheter 21 is curved is avoided. As such, rotation of the adjustment catheter 21, and the adjustment housing 92 and its associated components attached to the adjustment catheter, due to whipping effects may be avoided whilst elongation of the adjustment catheter 21 may be resisted when a tensile force is applied to the adjustment catheter 21.

FIG. 7 shows an alternative arrangements of the adjustment and cutting features of the catheter device 12, together with features of the papillary anchor 19. The arrangement shown holds the line clamping mechanism in an open position, fixes the papillary anchor 9 to the adjustment catheter 21 and provides a way to cut excess line 14.

An internal cam 91 may hold the papillary anchor locking segment 28 in an open position, i.e. with the slit open, and the cam 91 advantageously performs several tasks at the same time. The cam 91 can open the slit of the locking segment 28 as well as fixing the papillary anchor 9 to the adjustment catheter 21. In addition a cutting section 94 can be fitted to the cam wedge 95 that holds the cam 91 in the open position allowing the excess artificial chordae line 14 to be cut in the same movement. This reduces the need for wires going through the adjustment catheter 21. The cam 91 is held in place and supported by a holder that prevents the cam from twisting and or bending when actuated. The adjustment housing 92 may have protruding features or an interference fit around its perimeter that snaps in place with support brackets inside the distal part of the device, to allow the adjustment catheter 21 to extended the flexible and adjustable joint 34 then push out the papillary anchor 19, once the right amount of counter-pressure is exerted by tissue on the distal part 8 of the catheter device.

In this example the papillary anchor 19 locking segment 28 is held open with an internal cam 91. The cam 91 has a rest position (not shown) and one open position, as shown in FIG. 30, with the cam 91 in its open position the papillary anchor 19, and locking segment 28 are held open by internally applying a constraining force. The cam 91 is held in place by a housing 92 that supports the cam 91 structurally during its travel. In addition the adjustment housing 92 contains a line channel 93 and a sliding channel 99 for a combined cutting and cam wedge piece 96/95. When the cam wedge 95 is engaged with the cam's wedge-grooves 98 the anchor locking ring 28 is held open, the artificial chordae line 14 may then be threaded through the line channel 93 and through the open locking rings 28 with relatively free passage. Once a wire 97 connected through attachment hole 100 in the cutting wedge 96 is pulled, the wedge 95 disengages from the wedge-grooves 98 and the cam 91 returns to its rest position, clamping the line 14 and releasing the papillary anchor 19 from the adjustment housing 92. During the release of the cam 91, or immediately after, the cutting knife 94 engages with the line 14. The cam 91 and cutting wedge 96 may have a cylindrical shape, to accommodate tight tolerance machining. One or both of the cutting edges may also be fitted with flat or circular blades. An additional two legged fork structure (not shown in FIG. 7, but similar to that illustrated in FIGS. 5 and 6) connected to the wedge 96 that holds locking segment 28 open may also be included to make sure the locking segment 28 of the anchor is completely open while the suture 14 is adjusted.

Again, the catheter 1 can be used as the adjustment catheter 21. The catheter 1 can be joined, such as via welding, to the cutting section 94 or a proximal end thereof, such that the cutting section 94 of the cutting wedge 96 can be operated using the adjustment catheter 21. Additionally or alternatively, the adjustment catheter may house the piston wire 97 and/or any other wires for operating the adjustment and cutting features associated with deployment of the papillary anchor 19.

A steerable introducer with both a curving and a twisting capability is shown in various configurations in FIGS. 8 to 11. The introducer has a distal end 210 and a main body 212 (not shown in full). A flexible distal portion 214 is provided at the end of the main body 212, with this flexible distal portion 214 allowing for movement of the distal end 210 using varying degrees of curvature as shown in FIG. 8 and FIG. 9, and also with the addition of a twisting deformation as shown in FIG. 10 and FIG. 11. A catheter device, such as that of WO 2020/109599 and as discussed above, or the device of WO 2016/042022, can be housed in a hollow within the flexible distal portion 214, with this hollow also extending into the main body 212.

The steerable introducer may be used to introduce such a catheter device into the heart, for example in order to perform a procedure to implant an artificial heart chord as discussed in WO 2020/109599 and as discussed above, or as discussed in WO 2016/042022. The curvature as shown in FIGS. 8 and 9 can be used to steer the introducer as it passes through blood vessels as well as to locate the distal end 210 at a required position in the heart, for example to direct the catheter device to a required location in the heart when it is deployed from the distal end 210. The twist of the flexible distal portion 214 can be used to more accurately place the distal end 210 with reference to lateral movement such as a movement between the position in FIG. 10 and that in FIG. 11. In addition the ability to twist the flexible distal portion 214 allows for better adjustment of the angle of the distal end 210. It will be seen that in FIG. 10 the distal end 210 is angled differently compared to the angle of the distal end 210 in FIG. 11.

The combined steering and twisting capability is obtained via an arrangement of control wires 216, 218, 218′ along with an elastic element 220 within the flexible distal portion 214, acting as a form of “backbone”. As shown in FIGS. 12, 13 and 14 the elastic element 220 in an example embodiment is formed of a flexible tube 220 with cuts 222 along its length to provide a required flexibility. The tube 220 may for example be formed from a suitably elastic metal such as nitinol or stainless steel, and/or from a fibre reinforced polymer material, such as PEEK. This example uses a pair of twisting control wires 16 as well as two steering control wires 218, 218′. There is a primary steering control wire 218 that extends to a distal end thereof at first point 223 along the length of the flexible distal portion 214 as well as a secondary steering control wire 218′ that extends to a distal end thereof a different point 223′ along the length of the flexible distal portion 214, as shown in FIG. 13. The twisting control wires 216 have their distal ends fixed to the flexible distal portion 214 at a second position 224 and a third position 226 at the distal end 210 of the elastic element 220. The first, second and third positions are at different points around the circumference of the flexible distal portion 214 in order to achieve the required control of the curvature and twist thereof. The second position 224 and the third position 226 are at the same point along the axis of the flexible distal portion 214, and are symmetrically spaced either side of the first position 223.

The flexible distal portion 214 is provided with a first flexible section 228 and a second flexible section 230 with different elastic characteristics. The different elastic characteristics may arise in part due to differences in the elastic element 220, which has a corresponding first flexible section 228 and a second flexible section 230. The differences in the elastic element 220 may be differences in material type of thickness. For example the first flexible section 228 may comprise a fibre reinforced polymer and the second flexible section 230 may comprise a metal material. The first flexible section 228 and second flexible section 230 may be formed with cuts 222 of different geometry to provide differing behaviours for the first flexible section 28 and the second flexible section 230 when forces are applied by the control wires 216, 218, 218′. It will be appreciated that the design of the cuts can be varied from that shown whilst achieving a similar effect. In this example the first flexible section 228 is able to flex in all lateral directions, i.e. side to side as well as front to back, since the cuts 222 in the first flexible section 228 provide openings at all points around the circumference. In contrast, the second flexible section 230 has cuts 222 providing openings along the two sides as shown in FIGS. 12 and 13 with a stiffener 231 along the front and back. The stiffener 231 in this example is an uncut portion of the tube running along the length of the second flexible section 230. This makes the second flexible section 230 relatively rigid in relation to bending forces applied in a front to back direction, but relatively flexible in relation to side to side flexing, i.e. into and out of the page with reference to FIGS. 12 and 13.

It will be understood that an increased tension in the steering control wires 218, 218′ will tend to reduce the length of the second flexible section 230 and hence impart a curvature to the flexible distal portion 214 as shown in FIGS. 8 and 9. Since the two steering control wires 218, 218′ have differing lengths then it is possible to have a varying curvature along the length of the flexible distal portion as shown in FIG. 9. In addition it is possible to have a panning motion of the distal end 210 with a consistent angle of the distal end. The twisting control wires 216 are used to impart a twist to the flexible distal portion 214 and in particular to deform the first flexible section 228, as shown in FIGS. 10 and 11. Varying the relative tension in the two twisting control wires 216 will change the degree of twist. It should be noted that the resting position for the elastic element 220 may not be the straight position as shown in FIGS. 12, 13 and 14. Instead it may be heat set into a deformed position, which is advantageously a position part way toward an intended position for deployment of the catheter device. This deployment position could be similar to that of FIG. 9 for example, and the resting position of the elastic element 220 may be 40% deformed toward such a position. The flexibility of the cutting pattern can also allow for changes in shape and diameter as the catheter device passes through within the hollow 235, which can be seen in FIGS. 16 and 17.

The elastic element 220 can be encased in an elastic material, for example an elastic polymer, which may hence comprise elastic material 232 in the cuts 222 as shown in Figure as well as elastic material 234 surrounding the elastic element 220 as seen in the cross-sectional view of FIG. 16. The elastic material 232, 234 may be a compressible material allowing for deformation with minimal buckling. The composition and/or thickness of the elastic material 232, 234 may vary along the length of the flexible distal portion to vary the flexibility at different points, in particular to contribute to a different elasticity for the first flexible section 228 and the second flexible section 230.

The positions of the control wires 216, 218, 218′ at the circumference of the elastic element 220 can be seen FIGS. 16 and 17. FIG. 16 shows the elastic material 234 provided around the outside of the flexible distal portion 214. FIG. 17 shows a flexible coil 236 wrapped around the elastic element 220, and this is also shown in FIG. 18. A flexible coil 236 can optionally be used to add stiffness and to protect the flexible distal portion 214. The coil 236 and the elastic material 232, 234 can be used together. In this example the control wires 216, 218, 218′ are placed outside of the elastic element 220, inside the coil 236, and embedded in the elastic material 234. The control wires 216, 218, 218′ could alternatively be within the elastic element 220 or outside the coil 236. There may be additional steering control wires for further refinement of the steering control action. Another option is for a straightening wire to modulate the curve.

The catheter 1 can be introduced into the body by the steerable introducer, via the hollow 235 of the steerable introducer. The curving and twisting of the steerable introducer can actuate the catheter 1 such that the catheter too is twisted or curved. Each of these motions may cause extensions and/or result in the application of a tensile force on the catheter 1. By providing a catheter 1 comprising a tensile member 3 including a first portion 3a and a second portion 3b, 3b′, 3b″, 3b′″, the onset of whipping stresses induced due to an extension of the tensile member 3 when the catheter 1 is curved by the steerable introducer is avoided. As such undesirable rotation of the catheter 1 may be avoided, which could cause sudden movements and/or displacement of the steerable introducer potentially resulting in trauma or damage to the body during insertion, whilst elongation of the catheter 1 may be resisted when a tensile force is applied to the catheter 1.

1. A catheter for insertion into the body, the catheter comprising:
a flexible body portion having a longitudinal axis extending from a proximal end of the catheter towards a distal end of the catheter; and
a tensile member comprising a first portion extending along a first length of the tensile member and a second portion extending along a second length of the tensile member, the tensile member in communication with and extending along an outer part of the flexible body portion;
wherein the tensile member is configured to follow a curvature of the flexible body portion when the catheter is curved;
wherein the first portion is configured to increase a longitudinal stiffness of the catheter; and
wherein the second portion is slack along its length when the flexible body portion is in the unconstrained position, such that a longitudinal stiffness of the second portion is reduced relative to the first portion until the slack has been taken up through flexing of the catheter.

2. A catheter as claimed in claim 1, wherein the first portion extends along a majority of the tensile member, and wherein the second portion is located toward the distal end of the catheter compared to the first portion.

3. A catheter as claimed in claim 1 or 2, wherein the tensile member is a braided material, and wherein the second portion comprises a loosened portion of braid.

4. A catheter as claimed in claim 3, wherein the loosened portion of braid comprises a longitudinally compressed portion of the braided material.

5. A catheter as claimed in any preceding claim, wherein the second portion comprises a series of undulations extending circumferentially in relation to the flexible body portion.

6. A catheter as claimed in claim 5, wherein the undulations are shaped in a wave pattern, optionally wherein the wave pattern is at least one of: a saw wave, a triangular wave or a sinusoidal wave.

7. A catheter as claimed in any of claims 1 to 6, wherein the second portion of the tensile member is configured to increase in length without tensile force when the catheter is curved such that a rotation of the catheter is avoided.

8. A catheter as claimed in any of claims 1 to 7, wherein the longitudinal stiffness of the second portion is reduced relative to the first portion such that a rotation of the catheter is avoided when the catheter is curved and when the tensile member follows a radius of curvature greater than an inner radius of curvature of the flexible body portion.

9. A catheter as claimed in any preceding claim, wherein the tensile member is formed of Kevlar braid.

10. A catheter as claimed in any preceding claim, wherein the flexible body portion and the tensile member are embedded in a polymer.

11. A catheter as claimed in any preceding claim, wherein a distal end of the tensile member is fixed at a distal end of the flexible body portion, and wherein a proximal end of the tensile member is fixed at a proximal end of the flexible body portion.

12. A catheter device for implanting an anchor into body tissue to attach a line to the body tissue, the catheter device comprising:
an anchor for implantation in the body tissue to hold a line; and
a catheter as claimed in any of claims 1 to 11.

13. A catheter device as claimed in claim 12, the catheter device comprising:
an anchor holder that connects to the anchor whilst it is stowed and during deployment and releases the anchor after successful deployment of the anchor;
wherein the anchor holder comprises a piston for engagement with the anchor and a piston housing for holding the piston, with the piston able to be actuated for sliding movement relative to the piston housing; and
wherein a proximal end of the piston is attached to a distal end of the catheter.

14. A catheter device as claimed in claim 13, wherein the piston includes a cutter for cutting of the line once the anchor has been successfully deployed in a desired location, with the line adjusted to a suitable length.

15. A catheter device as claimed in claim 12, 13 or 14, the catheter device comprising:
a housing section extending from a distal end of the catheter device along the length of the catheter device toward the proximal end of the catheter device, the housing section comprising a distal part at the distal end of the catheter device and a proximal part located on the proximal side of the distal part; and
an anchor deployment mechanism at the distal part of the housing section for deployment of the anchor for attachment of the anchor to the body tissue, wherein the anchor deployment mechanism is arranged for deployment of the anchor from a stowed position of the anchor by moving it outward in the distal direction relative to the distal part;
wherein the anchor is held in its stowed position by the anchor deployment mechanism in the distal part prior to deployment
wherein the anchor deployment mechanism comprises an adjustment housing that holds the anchor during deployment and facilitates adjustment of the line; and
wherein the adjustment housing forms the anchor holder.

16. A catheter as claimed in any of claims 12 to 15, wherein the anchor comprises a number of hooks for engagement with the body tissue and having a folded position and an unfolded position, wherein the anchor is made of an elastic material such that the hooks can be elastically deformed into the folded position by application of a constraining force, and will return to the unfolded position when no constraining force is applied, and wherein the hooks are held in the folded position whilst the anchor is in the stowed position within the distal part.

17. A catheter device as claimed in any of claims 12 to 16, wherein the catheter is axially offset from a longitudinal axis of the catheter device, such that rotation of the catheter device about its longitudinal axis will rotate the catheter when the catheter device is in an unconstrained position.

18. A catheter device as claimed in claim 17, wherein the tensile member is configured to asymmetrically increase a longitudinal stiffness of the catheter such that when the catheter device is rotated while curved the longitudinal axis of the catheter will align with the longitudinal axis of the catheter device; and
wherein the catheter device is configured to rotate independently of the catheter when the longitudinal axes of the catheter device and the catheter are aligned.

19. A steerable introducer for a catheter device, the steerable introducer comprising:
a body portion having a longitudinal axis extending from a proximal end of the introducer toward a distal end of the introducer, wherein the body portion comprises a flexible distal portion extending to the distal end;
a hollow within a part of the body portion holding the catheter of any of claims 1 to 11; and
at least one steering control wire extending axially along the body portion and along at least a part of an axial length of the flexible distal portion;
wherein the at least one steering control wire has a distal end that is fixed to the flexible distal portion at a first point in a first axial and circumferential position on the flexible distal portion, with the introducer being arranged to allow for tension on the at least one steering control wire to cause a curvature of the flexible distal portion; and
wherein the catheter is configured to follow the curvature of the flexible distal portion.

20. A method of use of the catheter as claimed in any of any of claims 1 to 11 for implanting an anchor into body tissue for implanting an artificial chordae line, the method comprising:
inserting the catheter into the body.

21. A method of manufacture of the catheter as claimed in any of claims 1 to 11, the method comprising:
embedding the tensile member and the flexible body portion in a soft polymer.

22. A method of manufacture of the catheter as claimed in claim 21, wherein the soft polymer comprises Pebax, and wherein the step of embedding is performed using Pebax reflow.
